(19) United States
(12) Patent Application Publication
(10) Pub. No.: US 2003/0153808 A1
(43) Pub. Date: Aug. 14, 2003

(54) Implantation rate using ff-mas

(76) Inventor: Christa Hegele-Hartung, Muelheim/Ruhr (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/220,025

(22) PCT Filed: Feb. 15, 2001
(86) PCT No.: PCT/EP01/01692

(30) Foreign Application Priority Data
Feb. 25, 2000 (EP) 00250068.4

(51) Int. Cl. A61D 7/00; A61B 17/43

(52) U.S. Cl. 600/33

(57) ABSTRACT

The present invention relates to the use of FF-MAS and/or its analogues to increase the implantation rate of preimplantational embryos.

The present invention relates to a method of increasing the implantation rate of preimplantational embryos.

Fertilization of the mammalian egg is preceded by the maturation of the oocyte. This maturation process in mammalian oocytes includes nuclear and cytoplasmic changes. The first meiotic division from prophase I to metaphase II is reinitiated and completed. Only little is known about the mechanism which controls the initiation of meiosis. The presence of a diffusible meiosis regulating substance was first described by Byskov et al. in a culture system of fetal mouse gonads [Byskov, A. G. et al. Dev Biol 52 (1976) 193-200]. A meiosis activating substance (MAS) was secreted by the fetal mouse ovary in which meiosis was ongoing, and a meiosis preventing substance (MPS) was released from the morphologically differentiated testis with resting, non-meiotic germ cells. It was suggested that the relative concentrations of MAS and MPS regulated the beginning, arrest and resumption of meiosis in the male and in the female germ cells (Byskov, A. G. et al. in The Physiology of Reproduction [eds. Knobil. E. and Neill, J. D., Raven Press, New York (1994)]. Certain sterols (T-MAS and FF-MAS) that activate oocyte meiosis have been isolated from bull testes and from human follicular fluid [Byskov, A. G. et al. Nature 374 (1995), 559-562].

In in vitro feritilization (IVF) experiments with mouse oocytes, FF-MAS has been shown to increase the fertilization rate when added to the culture medium (Hegele-Hartung, C et al., Hum Reprod 13(suppl): 98 (1998)). The first clinical data with human oocytes showed an improvement in nuclear and cytoplasmic maturation(Grøndahl, C et al, 1999, Proceedings from The Second International Alpha Congress). Unfortunately, FF-MAS is rather labile. Therefore, stable synthetic analogues were developed. Such compounds being known to regulate the meiosis are described in the International patent applications WO 96/00235, WO 96/27658, WO97/00884, WO98/28323,WO98/52965 and WO 98/55498.

After fertilization of the oocytes and the development of the pre-embryo, the pre-embryo has to implant in the endometrium. The implantation rate of the IVF procedure used at present is relatively low. Only about 15-25% of the preimplantational embryos implant. Therefore, it would be desirous to have a method to improve the implantation rate after the IVF procedure.

The cause of infertility of women is also quite often the low implantation rate of the preimplantational embryo. Therefore, an in vivo treatment is needed which improves the implantation rate.

The present invention provides methods to increase the implantation rate of preimplantational embryos in females, preferably humans. An object of the present invention is the use of FF-MAS and/or one or more FF-MAS analogues to increase implantation rate of preimplantational embryos.

FF-MAS is 4,4-dimethyl-5a-cholesta-8,14,24-triene-3β-ol. Its synthesis is described in WO99/52930. FF-MAS analogues and their synthesis are described in e.g. WO 96/00235, WO 96/27658, WO97/00884, WO98/28323,WO98/52965 and WO 98/55498. FF-MAS analogues have a percentage germinal vesicle breakdown (GVB) which is significantly higher than the control. Preferred FF-MAS analogues are such having a percentage GVB of at least 50%, preferably at least 80%. Implantation rate means the rate by which a preimplantational embryo is able to interact with the endometrium thereby giving rise to the development of a postimplantational embryo and a fetus.

FF-MAS and/or one or more FF-MAS analogues can be added to a medium used in in vitro fertilization. The IVF procedure is performed in a known manner. More details about the removal of the oocytes from follicles in the ovary, culturing of the isolated oocytes, the culture medium to be used, the fertilisation with sperm and the transfer of the embryo to the fallopian tube can be found in the literature, e.g. in U.S. Pat. No. 5,693,534 which is hereby incorporated by reference. When FF-MAS and/or one or more FF-MAS analogues is added to the IVF medium, the implantation rate of the transfered embryo is increased by at least 20%, preferably by at least 50%. The concentration of FF-MAS and/or one or more FF-MAS analogues may be in the range of about 0.1-100 μmol/l.

FF-MAS and/or one or more FF-MAS analogues can also be used in vivo by applying it/them to females. FF-MAS and/or one or more FF-MAS analogues can be used for the preparation of a medicament that increases the implantation rate of oocytes. When given to a woman before and during ovulation, FF-MAS and its analogues increase the implantation rate by at least 20%. More preferably, the implantation rate is increased by at least 50%.

Due to the increase in the implantation rate, the pregnancy rate will also be higher. The medicament of this invention can therefore be used to treat females who have problems with infertility.

It may also be used to increase the implantation rate in animals which is important in animal breeding.

A further object of the present invention are pharmaceutical compositions comprising FF-MAS and/or one or more FF-MAS analogues. The compositions may further comprise pharmaceutically acceptable excipients well known in the art like carriers, diluents, absorption enhancers, preservatives, buffers, agents for adjusting the osmotic pressure, tablet disintegrating agents and other ingredients which are conventionally used in the art.

Examples of solid carriers are magnesium carbonate, magnesium stearate, dextrin, lactose, sugar, talc, gelatin, pectin, tragacanth, methylcellulose, sodium carboxymethyl cellulose, low melting waxes and cacao butter.

Liquid compositions include sterile solutions, suspensions and emulsions. Such liquid compositions may be suitable for injection. The liquid compositions may contain other ingredients which are conventionally used in the art, some of which are mentioned in the list above. Further, a composition for transdermal administration of FF-MAS and/or one or more FF-MAS analogues may be provided in the form of a patch and a composition for nasal administraton may be provided in the form of a nasal spray in liquid or powder form.

The dose of FF-MAS and/or one or more FF-MAS analogues to be used will be determined by a physician and will depend among several factors on the particular compound employed and on the route of administration. In general, the compositions of the invention are prepared by intimately bringing into association the active compound with the liquid or solid auxiliary ingredients and then, if necessary, shaping the product into the desired formulation.

Usually, not more than 1000 mg, preferably not more than 100 mg, and in some preferred instances not more than 10 mg of FF-MAS and/or one or more FF-MAS analogues is to be administered to mammals, e.g. to humans, per day. Treatment may either be continuously or intermittent. Intermittent treatment preferably starts before the ovulation and is continued until some days after ovulation.

The route of administration of FF-MAS and/or one or more FF-MAS analogues may be any route which brings the active compound(s) to its or their site of action. Examples for routes of administration are intravenous, subcutaneous, oral, intranasal or transdermal administration.

The present invention will be illustrated in detail in the following examples.

### EXAMPLE 1

### Method Used for Electing MAS Compounds

Oocytes were obtained from immature female mice (C57BI/6J×DBA/2J F1-hybrids, Bomholtgaard, Denmark) weighing 13-16 grams, that were kept under controlled lighting and temperature. The mice received an intra-peritoneal injection of 0.2 ml gonadotropins (Gonal F, Serono, Solna, Sweden, containing 20 IU FSH, alternatively, Puregon, Organon, Swords, Ireland containing 20 IU FSH) and 48 hours later the animals were killed by cervical dislocation.

The ovaries were dissected out and the oocytes were isolated in Hx-medium (see below) under a stereo microscope by manual rupture of the follicles using a pair of 27 gauge needles. Spherical, naked oocytes (NO) displaying an intact germinal vesicle (GV) were placed in α-minimum essential medium (α-MEM without ribonucleosides, Gibco BRL, Cat.No. 22561) supplemented with 3 mM hypoxanthine (Sigma Cat. No. H-9377), 8 mg/ml Human Serum Albumin (HSA, State Serum Institute, Denmark), 0,23 mM pyrubate (Sigma, Cat. No. S-8636), 2 mM glutamine (Flow Cat. No. 16-801), 100 IU/ml penicillin and 100 μg/ml streptomycin (Flow, Cat No. 16-700). This medium was designated Hx-medium. The oocytes were rinsed three times in Hx-medium and cultured in 4-well multidishes (Nuncion, Denmark) in which each well contained 0.4 ml of Hx-medium and 35 - 45 oocytes. One control (i.e. 35-45 oocytes cultured in Hx-medium with no addition of test compound) was always run simultaneously with the test cultures, which were made with different concentrations of the compounds to be tested. The cultures were performed at 37° C. and 100% humidity with 5% CO₂ in air. The culture time was 22-24 hours.

By the end of the culture period, the number of oocytes with germinal vesicle (GV) or germinal vesicle breakdown (GVB) and those with polar body (PB) was counted using a stereo microscope or an inverted microscope with differential interference contrast equipment. The percentage of oocytes with GVB per total number of oocytes and the percentage of oocytes with PB per total number of oocytes was calculated in the test cultures and compared to the control culture.

%GVB=((number of GVB+number of PB)/total number of oocytes)×100

### EXAMPLE 2

### Improvement of Embryo Implantation with FF-MAS and FF-MAS Succinate in the Cycling Rat

A total of 15 sexually mature female Wistar rats with a body weight of 200-220 g and a 4-day cycle were used. The animals were divided in one control group (group 1) and two treatment groups (group 2-3) consisting of 5 animals/group, respectively.

Starting in metestrus (d1) animals were treated for eight days (d1-d8), once daily, subcutaneously with the test compounds (treatment groups) or vehicle (control group), respectively. At day 7, at proestrus, animals were mated. The success of mating was checked by the presence of sperms in the vagina at d8. At d16 animals were sacrificed. Uteri were removed and checked for the presence of implantation sides. In each animal the number of implantation sides were determined.

Control group 1 received vehicle alone [(ethanol/arachisoil, 1+9 (v/v)].

Group 2 received 20 mg/kg FF-MAS, prepared in an ethanol/arachisoil vehicle.

Group 2 received 20 mg/kg FF-MAS-succinate, prepared in an ethanol/arachisoil vehicle.

The results are presented in Table 1.

TABLE 1
Effect of subcutaneous FF-MAS and FF-MAS-succinate treatment on
implantation rate in the adult, cycling rat

| | Treatment schedule | | Number of implantation sides |
|---|---|---|---|
| | d1*-d8 | d7 | [x ± sd] |
| Group | Treatment [s.c.] | Mating_{—} | d16 |
| 1 | Vehicle | + | 10 ± 4 |
| 2 | 20 mg/kg | + | 15 ± 3 |
| | FF-MAS | | |
| 3 | 20 mg/kg | + | 12 ± 2 |
| | FF-MAS- | | |
| | succinate | | |

*treatment starts at metestrus

### EXAMPLE 3

### Method to Determine Implantatation Rate after IVF

Mouse Oocyte Cultivation

Oocytes are obtained by puncture using a fine needle from the ovaries of mice primed with PMSG (pregnant mare serum gonadotrophin). The oocytes are cultivated for approx. 20 hours in a defined hypoxanthine-containing culture medium (Hx medium, see above) with FF-MAS (10 μM) (37° C.). The control oocytes are cultivated in defined culture medium without FF-MAS for the same period of time. Both the FF-MAS-oocytes and the control oocytes have begun meiosis maturation at the end of the culture period. All oocytes punctured on one day (from various mice) are pooled and distributed over the two study groups.

In vitro-Fertilization (IVF) in Mice

The oocytes cultivated in the mouse oocyte assay are further processed in this experiment. They are washed briefly 3× in a defined medium not containing FF-MAS and subsequently transferred into an IVF medium not containing FF-MAS. Then a defined number of capacitated sperms (obtained from the epididymides of a male mouse) is added and the oocytes are incubated with the sperms for approx. 4 hours in an incubator at 37° C. Following incubation the number of inseminated oocytes is counted.

Tubal Retransfer in Mice

1 day before the retransfer female recipient mice (foster mothers) in pre-estrus are mated with vasectomized males in order to trigger pseudogestation. One day later a vaginal check is performed. Animals with positive, sperm-free vaginal prop (successful mating) enter the study. This day is day 0 of gestation (=day 0 after the sterile copulation). Approx 10-15 inseminated oocytes are transferred into one tube (right) to the foster mothers on day 0 after the sterile copulation under anesthetization. The contralateral tube remains unused (check for pseudogestation). Only inseminated oocytes from the IVF experiment are used.

Retransfer is repeated until approx. 20 evaluable litters (with viable fetuses) have been obtained in all study groups (10 μM FF-MAS and one untreated control group).

The foster mothers are killed on day 17 of gestation. Uteri are removed. The number of viable and dead fetuses, the number of foster mothers, the number of pregnant foster mothers and the number of pregnant foster mothers with viable fetuses and with total fetal death, respectively, are counted.

The results are given in Table 2.

TABLE 2
Retransfer of 2-cell embryos to foster mothers after treatment of mouse
oocytes with FF-MAS and subsequent in vitro-fertilization.

| | | | | | | Number of | | Numbers of | |
|---|---|---|---|---|---|---|---|---|---|
| | | | | | | pregnant | | pregnant | |
| | Concentration | | | Number of | | foster | | foster | |
| | of FF- | Number of | Number of | pregnant | | mothers | | mothers | |
| | MAS in | viable | foster | foster | | with viable | | with total | |
| Group | culture | fetuses | mothers | mothers | | fetuses | | fetal death | |
| number | medium | [n] | [n] | [n] | [%] | [n] | [%] | [n] | [%] |
| 1 | — | 45 | 153 | 43 | 28 | 20 | 47 | 23 | 54 |
| 2 | 10 μM | 42 | 69 | 27 | 40 | 21 | 78 | 6 | 22 |

1. Use of FF-MAS and/or one or more FF-MAS analogues to increase the implantation rate of preimplantational embryos

2. Use of claim 1 wherein FF-MAS and/or one or more FF-MAS analogues are added to a medium used in in vitro fertilization

3. Use of claim 1 wherein FF-MAS and/or one or more FF-MAS analogues are applied to females.

4. Use of FF-MAS and/or one or more FF-MAS analogues for the preparation of a medicament that increases the implantation rate of preimplantational embryos.

5. Use according to any of the claims 1-4 wherein the implantation rate is increased by at least 20%.
